(19) United States
(12) Patent Application Publication
(10) Pub. No.: US 2021/0332404 A1
(43) Pub. Date: Oct. 28, 2021

(54) LIPID NANOPARTICLES FOR MEASURING CHRONIC AND ACUTE RESPONSE TO EXERCISE

(71) Applicant: Northwestern University, Evanston, IL (US)

(72) Inventors: C. Shad Thaxton, Chicago, IL (US); Rohun U. Palekar, Chicago, IL (US)

(73) Assignee: Northwestern University, Evanston, IL (US)

(21) Appl. No.: 16/625,340

(22) PCT Filed: Jun. 21, 2018
(86) PCT No.: PCT/US2018/038819

§ 371 Date: Dec. 20, 2019

Related U.S. Application Data
(60) Provisional application No. 62/522,889, filed on Jun. 21, 2017.

(51) Int. Cl. C12Q 1/00; C12Q 1/48

(52) U.S. Cl. CPC C12Q 1/005; B82Y 15/00; C12Q 1/48

(57) ABSTRACT

The invention relates to biosensor for detecting and/or quantifying exercise and disease-risk associated enzymes in a biological sample and related methods. The biosensor includes a solid support and a nanostructure with a metallic core and a phospholipid shell capable of binding to a lecithin:cholesterol acyltransferase (LCAT) activator (BL-NP).

### RELATED APPLICATIONS

This application claims the benefit under 35 U.S.C. § 119(e) of U.S. provisional application No. 62/522,889, filed Jun. 21, 2017, which is incorporated by reference herein in its entirety.

### FEDERALLY SPONSORED RESEARCH

This invention was made with government support under FA8650-15-2-5518 awarded by the Air Force Research Laboratory. The government has certain rights in the invention.

### BACKGROUND OF THE INVENTION

Currently available molecular markers of health and disease are largely obtained at instances where individuals interface with medical professionals with access to clinical laboratories. Unfortunately, it is not currently possible for individuals to track molecular markers that may fluctuate over the short and long term in response to any number of factors like medication use, dietary changes, or exercise that may impact their overall “molecular fitness.” Such technology may revolutionize the way individuals take charge of their health.

The synthesis and applications of bio-inspired high-density lipoprotein nanoparticle (HDL NP) consisting of a 5 nm gold nanoparticle core serves as a scaffold for the assembly of 2-4 copies of apolipoprotein AI (apoAI) and a phospholipid bilayer, where the inner leaflet of the bilayer is covalently conjugated to the gold surface via a thiolated head group. The final HDL-NP constructs are ˜13 nm in diameter and consists of 3±1 apoAI molecules as 83±12 phospholipids in the outer leaflet of the HDL-NP membrane, which compares favorably to the reported values for native, mature spherical HDL.

### SUMMARY OF THE INVENTION

Interestingly, it has been demonstrated that the spontaneous assembly of apo A-I on HDL-NPs on gold nanoparticle templates of −5-6 nm in diameter are optimal. Even a slight increase in the nanoparticle size resulted in reduced apo AI assembly and drastically reduced function with regard to binding cholesterol. Thus, 5 nm diameter AuNPs surface functionalized with PL provide a platform for specific sequestration of apoAI, which supports LCAT activity. Because of the core gold nanoparticle, a plethora of rapid biosensor readout options are available for the development of next generation molecular fitness assays.

Cardiovascular disease, despite all forms of therapy and intervention, continues to be the most common cause of death in the developed world. Exercise is near universally recommended as an intervention to reduce the risk of cardiovascular disease and to improve overall health. Unfortunately, it is difficult for individuals to monitor the health benefits of exercise, especially at the molecular level, to assess chronic and acute benefits. This invention meets this unmet need by developing technology that can, ultimately, enable personalized monitoring of chronic and acute fluctuations of molecular markers that change due to exercise and, potentially, other beneficial diet, lifestyle, or pharmacologic interventions. We term this new era of patient-directed monitoring “molecular fitness.”

High-density lipoproteins (HDL) remove free cholesterol (C) from macrophages associated with atherosclerotic plaques. HDL-associated free cholesterol is esterified by lecithin:cholesterol acyl transferase (LCAT) in the presence of the HDL-specific cofactor apolipoprotein A-I (apoAI) in the serum, thereby forming cholesteryl esters (CE) in the core of growing HDL particles. Cholesterol-rich HDLs transport cholesterol to the liver for excretion. This process, termed reverse cholesterol transport (RCT), is believed to reduce the risk of cardiovascular disease.

5 nm diameter gold nanoparticles (AuNPs) surface-functionalized with phospholipids as templates have been utilized to assemble apoAI for HDL nanoparticles (HDL NP). To investigate the potential for rapid measurement of RCT parameters, like apoAI and LCAT activity, we utilized 5 nm diameter gold nanoparticles (NP) decorated with a phospholipid bilayer (BL). These BL-NPs rapidly and preferentially adsorb apoAI from pure solutions and human serum according to the abundance of apo AI in the sample. ApoAI coated PL NPs provide the co-factor (apoAI) and substrates, PL and free C, for LCAT activity. Thus, after isolation of the nanoparticles based upon apoAI, a colorimetric oxidase assay for C and CE was performed to quantify LCAT activity. The assay provides for rapid measurement of apoAI and LCAT activity, two key parameters of RCT.

In some aspects a biosensor for detecting and/or quantifying an exercise and disease-risk associated enzyme in a liquid sample is provided. The biosensor comprises (a) a solid support; and (b) a nanostructure comprising a metallic core of 1-10 nm in diameter and a phospholipid shell capable of binding to a lecithin:cholesterol acyltransferase (LCAT) activator (BL-NP), wherein the BL-NP is bound to the solid support.

In some embodiments the biosensor is a wearable device. In other embodiments the biosensor is not a wearable device.

In some embodiments the solid support comprises plastic, glass, metal, metal oxide or crystal, and the solid support is in the form of a testing strip, a well in a microwell plate or a microcentrifuge tube; or (b) the solid support comprises or consists essentially of silica gel or other porous particle packing material for chromatography.

In some embodiments the phospholipid shell binds the LCAT activator. In other embodiments the LCAT activator is an apolipoprotein.

In some embodiments the metallic core is a gold core. In other embodiments the gold core has a diameter of 5 nm. In yet other embodiments the gold core has a diameter of 4-6 nm or 5-8 nm.

In some aspects the invention is a method for rapid detection of an exercise and/or disease-risk associated enzyme. The method involves contacting a biological sample with a biosensor comprising: (a) a solid support; and (b) a nanostructure comprising a metallic core of 1-10 nm in diameter and a phospholipid shell capable of binding to a lecithin:cholesterol acyltransferase (LCAT) activator (BL-NP), wherein the BL-NP is bound to the solid support, incubating the BL-NP with the biological sample for at least 15 minutes so that the BL-NP can bind to the LCAT activator, measuring LCAT activation as an indicator of the presence of the exercise and/or disease associated enzyme in the biological sample.

In some embodiments the biological sample is selected from the group consisting of blood, blood matrix, serum, plasma, sputum, cerebrospinal fluid, breath condensate, saliva, and tears. In other embodiments the biological sample is serum and 1-2 uL of serum is used.

In other embodiments the LCAT activator is an apolipoprotein.

In some embodiments the disease associated enzyme is indicative of cardiovascular disease.

In some embodiments the quantity of cholesterol ester present in the biological sample is determined. In other embodiments the biosensor provides an assessment of HDL function.

In some embodiments the biological sample is isolated from a subject after exercise. In other embodiments the biological sample is isolated from a subject during exercise. In yet other embodiments the biological sample is isolated from a subject before exercise. In other embodiments the biological sample is isolated from a subject during a clinical trial.

The biosensor in some embodiments is a lateral flow assay system. In other embodiments the biosensor is a paper assay system.

In yet other embodiments the phospholipids of the biosensor are not fluorescently labeled.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D. Western blot of apoAI content on BL-NP following incubation in apoAI-doped PBS (FIG. 1A) and serum (FIG. 1B). Measurement of esterification on BL-NP using an Amplex Red cholesterol assay following incubation with apoAI/cholesterol/LCAT in PBS (FIG. 1C) and serum (FIG. 1D).

FIG. 2. In situ synthesis of HDL-NP.

FIG. 3. Correlation plot of apoAI content in human serum samples versus LCAT activity on in situ synthesized BL-NP.

FIG. 4. Correlation plot of apoAI content on BL-NP post-incubation vs. total serum apoAI content.

FIG. 5. Overview of HDL function, which is an integrative metabolic biomarker of human health and performance.

FIG. 6. Overview of sequestration of ApoAI on BL-NP.

FIG. 7. Overview of measurement of Cholesterol Esterification on in situ HDL-NP with Amplex Red and measurement of Cholesterol Efflux.

FIGS. 8A-8B. Results showing that HDL-NP support cholesterol loading and efflux from cells.

FIGS. 9A-9B. Western blots following incubation of ApoAI with BL-NP (FIG. 9A) and incubation of BL-NP similarly in serum (FIG. 9B), to observe the amount of apoAI associated with the nanoparticles.

FIGS. 10A-10B. Results showing that incubation of BL-NP with either a mixture of apoAI/cholesterol/LCAT (FIG. 10A) or serum (FIG. 10B) demonstrates a dose-dependent apoAI adsorption and corresponding LCAT activity.

FIG. 11. Correlation plots of Cohorts A and B, showing significant positive correlation between serum apoAI and HDL cholesterol efflux, which confirms the relationship between each metric.

FIG. 12. Correlation plot showing that cholesterol esters on IS-HDL NP correlate with concentration of large HDL.

### DETAILED DESCRIPTION OF THE INVENTION

The invention, in some aspects, is a biosensor for the rapid, inexpensive detection and quantitation of cholesterol esters from bodily samples, as an indicator of both LCAT activity and apoAI levels. Together these factors are predictive of serum cholesterol levels which may be used to assess cardiovascular disease risk.

As shown in the examples the following experimental setup demonstrated effective cholesterol ester detection. The phospholipid bilayer nanoparticles (BL-NPs) are mixed with a serum sample and the particles are isolated. The total amount of cholesterol on BL-NP were measured using an Amplex Red Cholesterol Assay, which utilizes cholesterol esterase to hydrolyze all cholesteryl esters into cholesterol, after which all cholesterol associated with our sample would be oxidized by cholesterol oxidase to generate hydrogen peroxide. In the presence of 10-acetyl-3,7-dihydroxyphenoxazine and horseradish peroxidase this generates a fluorescent product, resorufin, providing a fluorescent readout corresponding to the amount of cholesterol in the sample. To determine cholesteryl ester content, each sample of serum-incubated BL-NP were assayed for cholesterol with or without the presence of cholesterol esterase. The importance of this distinction is that is allows us to determine the amount of total cholesterol in the sample, along with the amount of cholesterol excluding the contribution from cholesteryl esters. The net difference between these values allows the user to give a measurement of BL-NP associated cholesteryl esters. Using this method, it was determined that incubating BL-NP at serum concentrations of 1% (i.e. 1 μl of serum in 100 μl of 250 nM BL-NP) is adequate for well-defined activity of LCAT on in situ synthesized HDL-NPs.

It was also demonstrated in the Examples that BL-NP could sequester apoAI from serum samples and detect natural variations in apoAI that correlate with measured cholesterol esterification on the BL-NP.

These findings have several important points. First, due to the fact that there is a significant positive correlation between the amount of cholesterol esterification on BL-NP following incubation with serum and the amount of apoAI content in serum, this demonstrates that the BL-NP is an effective biosensor for detecting variations in RCT (and thus, LCAT) activity. Secondly, the biosensor is able to assay some elements of RCT activity in a manner that is sensitive to apoAI and LCAT activity variations in serum as a result of external physiological factors, such as exercise. This has not readily been achieved before in a simple one step assay.

Thus, the technology takes advantage of size-specific 5 nm diameter lipid functionalized gold nanoparticles that spontaneously sequester apoAI from human serum samples obtained by finger pin prick (as little is 1-2 uL of serum required). Because of the presence of LCAT, that uses the cholesterol in serum and the sequestered apoAI as substrate and co-factor, respectively, the cholesterol is esterified on the now-formed HDL NP (5 nm diameter gold core, phospholipids, and apoAI) and the amount of formed cholesteryl ester can be quantified to reflect LCAT activity. Each of these reaction points, 1) apoAI loading and 2) LCAT activity can be measured in point-of-care diagnostic setups that facilitate individual and multiple use. The markers are important markers that increase chronically (apoAI) and acutely (LCAT activity) secondary to exercise.

The invention has many uses and advantages. For instance, the methods disclosed herein provide personal monitoring of biological factors, such as apolipoprotein A-I (apoAI) and LCAT activity, that are chronic and acute, respectively, markers that increase in response to exercise. The methods and biosensors disclosed herein may be used to assess long-term increases in apoAI levels in response to exercise and increases in LCAT activity after individual exercise events. Accordingly, the biosensors will provide a personalized way of setting goals, measuring success, and tracking progress. Overall, the benefits way well be realized in terms of reduced CVD, increased overall health, lower rates of type II diabetes mellitus, metabolic syndrome, and any other diseases where exercise, or other interventions, may impact apoAI and/or LCAT activity.

The biosensors may also be used in clinical trials to monitor levels of key enzymes, in athletic events by athletes to measure response to exercise of different duration, intensity, type (e.g. weight training versus cardio training like running), or to assess dietary changes and increases in apoAI and/or LCAT activity.

A biosensor, as used herein, refers to is an analytical device, used to detect an analyte, directly or indirectly. The biosensor of the invention is useful for detecting cholesterol levels in a bodily sampleby measuring cholesterol esters as a result of ApoAI binding and LCAT activity. The biosensor includes a support and a BL-NP for binding ApoAI from the biological sample. The biosensor may also include a physicochemical detector such that the signal can be generated and detected all in one device. Examples of physicochemical detector systems include optical, piezoelectric, electrochemical, electrochemiluminescence.

In some instances the biosensor is a lateral flow assay system. A lateral flow assay test strip is configured to receive a sample of interest at a sample receiving region and to provide for the sample to move laterally through a flow region by capillary action to a detection region, such that the sample is wicked laterally through the flow region from the sample receiving region to the detection region. Such a system typically includes a support which may be a membrane containing bound BL-NP. Lateral flow of a biological fluid such as serum containing ApoAI over the membrane can allow the ApoAI to bind to the BL-NP and thereby detect ApoAI levels which correlate to cholesterol levels.

The biosensor may also be a test strip assay device, such as a paper strip assay. A method of evaluating a sample for the presence of ApoAI may involve the step of placing the sample onto a sample receiving location of a test strip assay device comprising BL-NP detection reagents and obtaining a signal from the test strip assay device to evaluate the sample for the presence of the ApoAI.

The methods and biosensors disclosed herein have some distinct advantages such as enabling home use, while conventional technology is not compatible with home use. The biosensors may also be interfaced with currently available smart phone, and similar, technology. Additionally the inventive technology is more specific, in the case of LCAT activity, than the conventional LCAT assay that relies upon non-specific LCAT fluorescent substrates. A variety of different test strips are encompassed within the invention. The particular nature of a test strip employed in a given assay will depend on a number of parameters. Test strips of interest include, but are not limited to, analyte oxidizing signal producing system test strips, lateral flow assay test strips, etc.

High-density lipoproteins (HDL) are naturally-occurring nanoparticles that assemble dynamically in serum from phospholipids, apolipoproteins, and cholesterol. HDL is involved in reverse-cholesterol transport, and has been epidemiologically correlated with reduced incidences of cardiovascular disease.^{3, 4} Natural HDL is known to bind Scavenger Receptor type B-1 (SR-B1); SR-B1 mediates uptake of cholesteryl esters and the uptake and efflux free cholesterol. Cholesterol uptake has been shown to be critical for proliferation of several types of cancers, including lymphoma, prostate cancer, and breast cancer.

Thus, in some aspects the invention is a nanostructure composed of a nanostructure core of an inorganic material surrounded by a shell of a lipid layer, and a therapeutic agent associated with the shell. The nanostructure may also include a protein such as an apolipoprotein.

The shell may have an inner surface and an outer surface, such that the therapeutic agent and/or the apolipoprotein may be adsorbed on the outer shell and/or incorporated between the inner surface and outer surface of the shell.

Examples of nanostructures that can be used in the methods are described herein are now described. The structure (e.g., a synthetic structure or synthetic nanostructure) has a core and a shell surrounding the core. In embodiments in which the core is a nanostructure, the core includes a surface to which one or more components can be optionally attached. For instance, in some cases, core is a nanostructure surrounded by shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physiosorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. The core (e.g., a nanostructure core or a hollow core) may have a largest cross-sectional dimension (or, sometimes, a smallest cross-section dimension) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

The core may be formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species, that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, sytrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or, the amphiphilic material may include a hydrophobic group that points towards the core and a hydrophilic group that extends away from the core. Bilayers of each configuration can also be formed.

The structures described herein may also include one or more proteins, polypeptides and/or peptides (e.g., synthetic peptides, amphiphilic peptides). In one set of embodiments, the structures include proteins, polypeptides and/or peptides that can increase the rate of cholesterol transfer or the cholesterol-carrying capacity of the structures. The one or more proteins or peptides may be associated with the core (e.g., a surface of the core or embedded in the core), the shell (e.g., an inner and/or outer surface of the shell, and/or embedded in the shell), or both. Associations may include covalent or non-covalent interactions (e.g., hydrophobic and/or hydrophilic interactions, electrostatic interactions, van der Waals interactions).

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and are particularly useful for binding to the BL-NP structures described herein.

Lecithin-cholesterol acyltransferase is an enzyme which converts free cholesterol into cholesteryl ester (a more hydrophobic form of cholesterol). In naturally-occurring lipoproteins (e.g., HDL and LDL), cholesteryl ester is sequestered into the core of the lipoprotein, and causes the lipoprotein to change from a disk shape to a spherical shape. Thus, structures described herein interact with lecithin-cholesterol acyltransferase to correlate with HDL and LDL function.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell.

A variety of methods can be used to fabricate the nanostructures described herein. Examples of methods are provided in International Patent Publication No. WO/2009/131704, filed Apr. 24, 2009 and entitled, “Nanostructures Suitable for Sequestering Cholesterol and Other Molecules”, which is incorporated herein by reference in its entirety for all purposes.

As described herein, the inventive structures may be used in “pharmaceutical compositions” or “pharmaceutically acceptable” compositions, which comprise a therapeutically effective amount of one or more of the structures described herein, formulated together with one or more pharmaceutically acceptable carriers, additives, and/or diluents. The pharmaceutical compositions described herein may be useful for treating cancer or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The structures described herein may be orally administered, parenterally administered, subcutaneously administered, and/or intravenously administered. In certain embodiments, a structure or pharmaceutical preparation is administered orally. In other embodiments, the structure or pharmaceutical preparation is administered intravenously. Alternative routes of administration include sublingual, intramuscular, and transdermal administrations.

Pharmaceutical compositions described herein include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

The inventive compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. An inventive structure may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

Dosage forms for the topical or transdermal administration of a structure described herein include powders, sprays, ointments, pastes, foams, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a structure described herein to the body. Dissolving or dispersing the structure in the proper medium can make such dosage forms. Absorption enhancers can also be used to increase the flux of the structure across the skin. Either providing a rate controlling membrane or dispersing the structure in a polymer matrix or gel can control the rate of such flux.

Ophthalmic formulations, eye ointments, powders, solutions and the like, are also contemplated as being within the scope of this invention.

Pharmaceutical compositions described herein suitable for parenteral administration comprise one or more inventive structures in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Delivery systems suitable for use with structures and compositions described herein include time-release, delayed release, sustained release, or controlled release delivery systems, as described herein. Such systems may avoid repeated administrations of the structures in many cases, increasing convenience to the subject and the physician. Many types of release delivery systems are available and known to those of ordinary skill in the art. They include, for example, polymer based systems such as polylactic and/or polyglycolic acid, polyanhydrides, and polycaprolactone; nonpolymer systems that are lipid-based including sterols such as cholesterol, cholesterol esters, and fatty acids or neutral fats such as mono-, di- and triglycerides; hydrogel release systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The compositions may be as, for example, microspheres, hydrogels, polymeric reservoirs, cholesterol matrices, or polymeric systems. In some embodiments, the system may allow sustained or controlled release of the active compound to occur, for example, through control of the diffusion or erosion/degradation rate of the formulation. In addition, a pump-based hardware delivery system may be used in some embodiments. The structures and compositions described herein can also be combined (e.g., contained) with delivery devices such as syringes, pads, patches, tubes, films, MEMS-based devices, and implantable devices.

Use of a long-term release implant may be particularly suitable in some cases. “Long-term release,” as used herein, means that the implant is constructed and arranged to deliver therapeutic levels of the composition for at least about 30 or about 45 days, for at least about 60 or about 90 days, or even longer in some cases. Long-term release implants are well known to those of ordinary skill in the art, and include some of the release systems described above.

Injectable depot forms can be made by forming microencapsule matrices of the structures described herein in biodegradable polymers such as polylactide-polyglycolide. Depending on the ratio of structure to polymer, and the nature of the particular polymer employed, the rate of release of the structure can be controlled. Examples of other biodegradable polymers include poly(orthoesters) and poly(anhydrides).

When the structures described herein are administered as pharmaceuticals, to humans and animals, they can be given per se or as a pharmaceutical composition containing, for example, about 0.1% to about 99.5%, about 0.5% to about 90%, or the like, of structures in combination with a pharmaceutically acceptable carrier.

The administration may be localized (e.g., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be useful for some treatments because of the convenience to the patient as well as the dosing schedule.

Regardless of the route of administration selected, the structures described herein, which may be used in a suitable hydrated form, and/or the inventive pharmaceutical compositions, are formulated into pharmaceutically-acceptable dosage forms by conventional methods known to those of skill in the art.

The compositions described herein may be given in dosages, e.g., at the maximum amount while avoiding or minimizing any potentially detrimental side effects. The compositions can be administered in effective amounts, alone or in a combinations with other compounds. For example, when treating cancer, a composition may include the structures described herein and a cocktail of other compounds that can be used to treat cancer. When treating conditions associated with abnormal lipid levels, a composition may include the structures described herein and other compounds that can be used to reduce lipid levels (e.g., cholesterol lowering agents).

The phrase “therapeutically effective amount” as used herein means that amount of a material or composition comprising an inventive structure which is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of any one or more structures described herein may be from about 10 ng/kg of body weight to about 1000 mg/kg of body weight, and the frequency of administration may range from once a day to once a month. However, other dosage amounts and frequencies also may be used as the invention is not limited in this respect. A subject may be administered one or more structure described herein in an amount effective to treat one or more diseases or bodily conditions described herein.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular inventive structure employed, the route of administration, the time of administration, the rate of excretion or metabolism of the particular structure being employed, the duration of the treatment, other drugs, compounds and/or materials used in combination with the particular structure employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

In some embodiments, a structure or pharmaceutical composition described herein is provided to a subject chronically. Chronic treatments include any form of repeated administration for an extended period of time, such as repeated administrations for one or more months, between a month and a year, one or more years, or longer. In many embodiments, a chronic treatment involves administering a structure or pharmaceutical composition repeatedly over the life of the subject. For example, chronic treatments may involve regular administrations, for example one or more times a day, one or more times a week, or one or more times a month. In general, a suitable dose such as a daily dose of a structure described herein will be that amount of the structure that is the lowest dose effective to produce a therapeutic effect. Such an effective dose will generally depend upon the factors described above. Generally doses of the structures described herein for a patient, when used for the indicated effects, will range from about 0.0001 to about 100 mg per kg of body weight per day. The daily dosage may range from 0.001 to 50 mg of compound per kg of body weight, or from 0.01 to about 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms. For example, instructions and methods may include dosing regimens wherein specific doses of compositions, especially those including structures described herein having a particular size range, are administered at specific time intervals and specific doses to achieve reduction of cholesterol (or other lipids) and/or treatment of disease while reducing or avoiding adverse effects or unwanted effects.

While it is possible for a structure described herein to be administered alone, it may be administered as a pharmaceutical composition as described above. The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition, including those associated with abnormal lipid levels. The kits can further include a description of use of the compositions as discussed herein. The kit also can include instructions for use of a combination of two or more compositions described herein. Instructions also may be provided for administering the composition by any suitable technique, such as orally, intravenously, or via another known route of drug delivery.

The kits described herein may also contain one or more containers, which can contain components such as the structures, signaling entities, and/or biomolecules as described. The kits also may contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the particular inventive structure and the mode of use or administration. Suitable solvents for compositions are well known and are available in the literature.

The kit, in one set of embodiments, may comprise one or more containers such as vials, tubes, and the like, each of the containers comprising one of the separate elements to be used in the method. For example, one of the containers may comprise a positive control in the assay. Additionally, the kit may include containers for other components, for example, buffers useful in the assay.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1

HDL NP was applied as a biosensor for RCT activity to demonstrate external physiological factors altering the activity or expression of key components involved with RCT. It was hypothesized that by synthesizing BL-NP and using serum samples as a source of apoAI, variations in apoAI content, and thus variations in LCAT activity would be detected. The use of the in situ formed HDL-NP for assaying LCAT activity is ideal, as HDL-NP provide both apoAI (the primary activator of LCAT in serum) and a donor pool of phosphotidylcholine from the outer leaflet of the nanoparticle membrane for transesterification of acyl chains to cholesterol.

As many commercially available kits for assaying RCT activity (e.g. LCAT activity kits) take upwards of 24 hours to determine results and are relatively unspecific in their detection of enzyme activity due to the lack of substrates directly specific to LCAT and the lack of dual readout with regard to apo AI, it was aimed to provide a method of detecting RCT components (e.g. apoAI and LCAT activity). Herein, an approach is reported to assess RCT activity through spontaneous apoAI binding and then a functional measurement of cholesterol esterification by LCAT on the surface of HDL-mimicking nano-particles (HDL-NP) formed in situ.

The HDL-NP utilized in this work differs from the traditional synthesis of HDL-NP commonly employed in the lab.¹¹⁻¹³ The synthesis of phospholipid bilayer nanoparticles (BL-NP) began by initially incubating 5 nm gold nanoparticles with phospholipids to complete assembly of the structure. The lipids used were a) 1,2-dioleoyl-sn-glycero-3-phosphoethanolamine-N-[3-(2-pyridyldithio)propionate] (PDP-PE), a thiolated lipid that binds covalently to the surface of the AuNP to form the inner leaflet, b) 1,2-dipalmitoyl-sn-glycero-3-phosphocholine (DPPC) which will form the majority of the outer leaflet of the lipid bilayer. Following over-night incubation with the phospholipid mixture, the nanoparticles were purified through sequential centrifugation or tangential flow filtration.

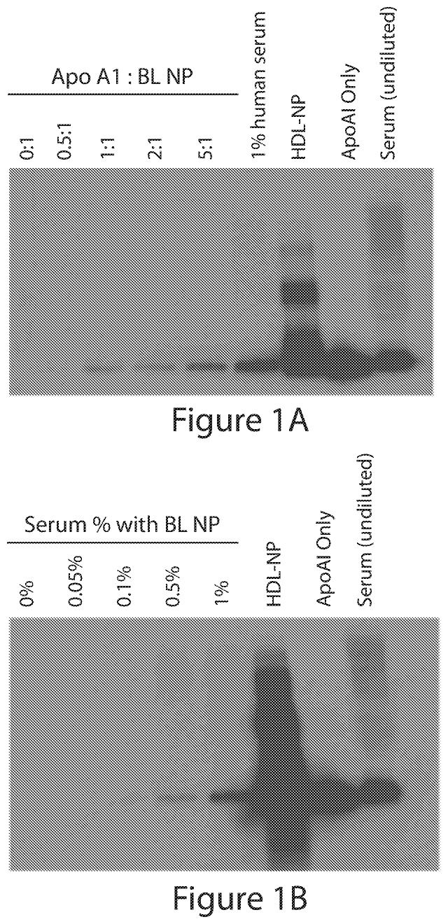

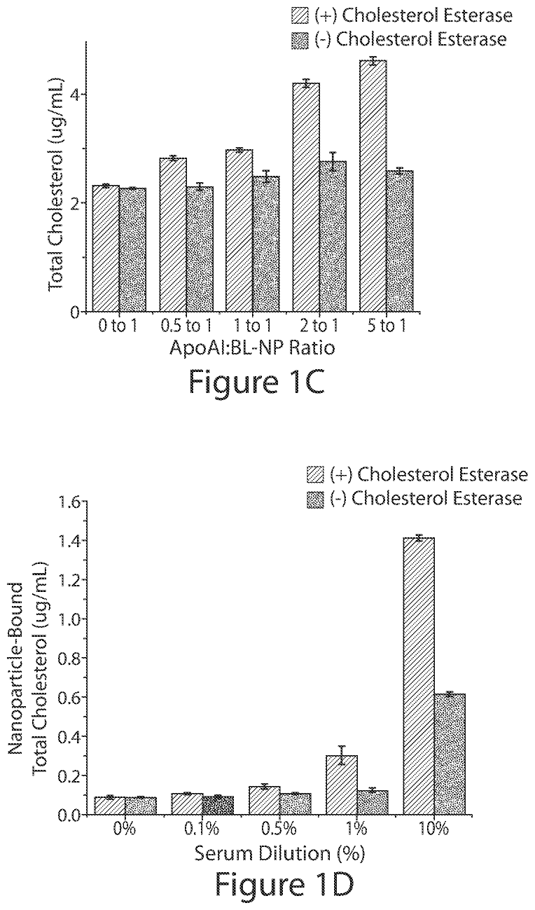

Initially, to measure the ability of BL-NPs to quantitatively sequester apo AI, a fixed concentration (250 nM) of NPs was incubated with either increasing amounts of apoAI in PBS, or serum at various concentrations to determine the ability of these nanoparticles to sequester apoAI from serum. Following 1 hour of incubation in either serum or an apoAI mixture, BL-NPs were isolated and purified using sequential centrifugation and probed for apoAI content through Western blotting (FIGS. 1A-1B) These results demonstrate a dose-dependent increase in BL-NP associated apoAI, confirming the ability of BL-NP to sequester apoAI from serum, essentially accomplishing an in situ synthesis of HDL-NP (FIG. 2. It was confirmed that the activity of these post-hoc HDL-NP to efflux cholesterol and compared them to a traditional synthesis of HDL-NP, and found they compared favorably to traditional HDL-NP (1.17% efflux for BL-NP+ApoAI vs. 1.36% efflux for HDL-NP).

It was also an aim to determine whether the sequestration of apoAI at various concentrations in the incubation medium would result in a concomitant variation in detected cholesteryl esters on BL-NP after exposure to LCAT (FIGS. 1C-1D). Once again, BL-NPs were incubated with increasing amounts of apoAI in PBS, or serum at various dilutions. For samples incubated with apoAI in PBS, cholesterol and LCAT were supplemented into the incubation medium. In this experimental setup, after particle isolation, the total amount of cholesterol on BL-NP were measured using an Amplex Red Cholesterol Assay, which utilizes cholesterol esterase to hydrolyze all cholesteryl esters into cholesterol, after which all cholesterol associated with our sample would be oxidized by cholesterol oxidase to generate hydrogen peroxide. In the presence of 10-acetyl-3,7-dihydroxyphenoxazine and horseradish peroxidase this generates a fluorescent product, resorufin, providing a fluorescent readout corresponding to the amount of cholesterol in the sample. To determine cholesteryl ester content, each sample of serum-incubated BL-NP were assayed for cholesterol with or without the presence of cholesterol esterase. The importance of this distinction is that is allows us to determine the amount of total cholesterol in the sample, along with the amount of cholesterol excluding the contribution from cholesteryl esters. The net difference between these values allows us to give a measurement of BL-NP associated cholesteryl esters. Using this method, it was determined that incubating BL-NP at serum concentrations of 1% (i.e. 1 μl of serum in 100 μl of 250 nM BL-NP) is adequate for well-defined activity of LCAT on in situ synthesized HDL-NPs.

TABLE 1
Characterization data for commercially obtained serum samples.

| Donor ID | 1 | 2 | 3 | 4 | 5 | 6 | 7 | 8 | 9 | 10 |
|---|---|---|---|---|---|---|---|---|---|---|
| Gender | F | M | M | F | M | F | M | M | M | F |
| Ethnicity | C | AA | H | C | C | AA | C | AA | C | H |
| Age (years) | 51 | 24 | 27 | 45 | 28 | 29 | 24 | 29 | 38 | 18 |
| [ApoA1] (mg/dL) | 116.056 | 84.042 | 105.708 | 120.222 | 115.431 | 138.625 | 132.167 | 115.569 | 132.514 | 118.764 |
| Total | 170.904 | 143.414 | 185.958 | 169.003 | 154.598 | 163.314 | 171.294 | 153.310 | 176.386 | 165.720 |
| Cholesterol | | | | | | | | | | |
| (mg/dL) | | | | | | | | | | |
| HDL-C (mg/dL) | 19.842 | 24.715 | 70.678 | 38.926 | 48.657 | 43.260 | 56.439 | 51.786 | 98.290 | 32.345 |
| CE (ug/mL) | 0.218 | 0.252 | 0.276 | 0.364 | 0.099 | 1.950 | 1.743 | 1.409 | 2.357 | 0.932 |

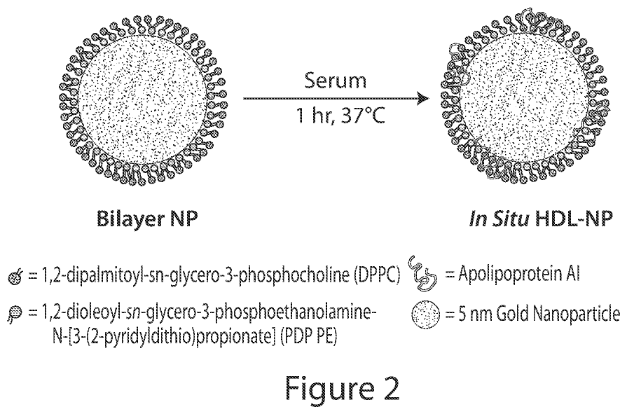

Next, it was sought to assess whether BL-NP could sequester apoAI from serum samples and whether natural variations in apoAI would correlate with measured cholesterol esterification on our nanoparticles. Commercially available serum samples were purchased from 10 de-identified human donors (Table 1) and characterized each serum sample for apoAI levels, total cholesterol, HDL-cholesterol (HDL-C), and the amount of CEs associated with BL-NP following a 1-hour incubation period with 1% serum. These measurement parameters were analyzed using the Pearson's correlation test. The results indicate that apoAI levels in serum are correlated (R=0.729, p=0.016, FIG. 3) with the number of CEs detected on the nanoparticles.

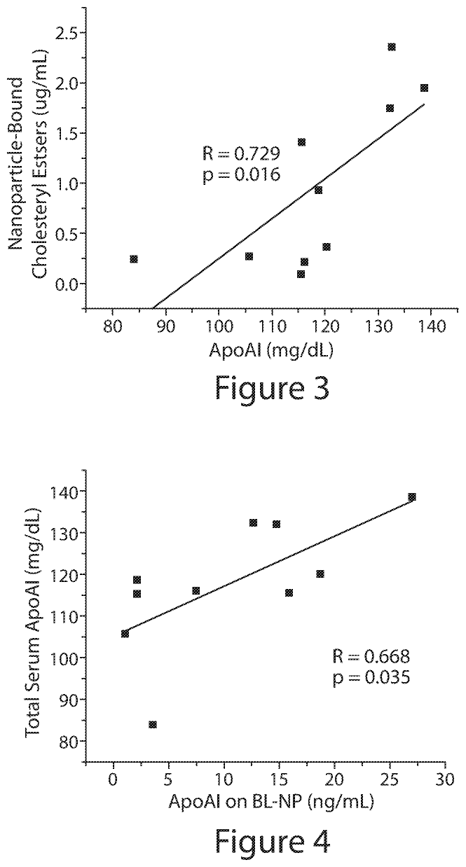

Furthermore, using an ELISA for apoAI, the ability of BL-NP to sequester variable amounts of apoAI from serum samples of differing total serum apoAI was quantified. The results shown in FIG. 4 depict a correlation plot between the total amount of apoAI in each of the aforementioned commercially-obtained serum samples and the amount of apoAI detected on BL-NP following a 1 hour incubation with 1% serum. These data demonstrate a significant positive correlation (R=0.668, p=0.035).

These results demonstrate two main points—first, due to the fact that there is a significant positive correlation between the amount of cholesterol esterification on BL-NP following incubation with serum and the amount of apoAI content in serum, this demonstrates the potential for BL-NP as a biosensor for detecting variations in RCT (and thus, LCAT) activity. Second, through the use of BL-NP, a substrate has been provided that allows for the sequestration of apoAI from the surrounding medium and that the extent of apoAI bound to the BL-NP is dependent on the total amount of apoAI in the medium, thus resulting in a post-hoc synthesis of HDL-mimicking nanoparticles, which in turn serve as a specific substrate for LCAT activity due to the presence of apoAI co-factor and substrates on the nanoparticle surface. This platform sets the foundation for a biosensor that would be able to assay some elements of RCT activity in a manner that is sensitive to apoAI and LCAT activity variations in serum as a result of external physiological factors, such as exercise.

### Example 2

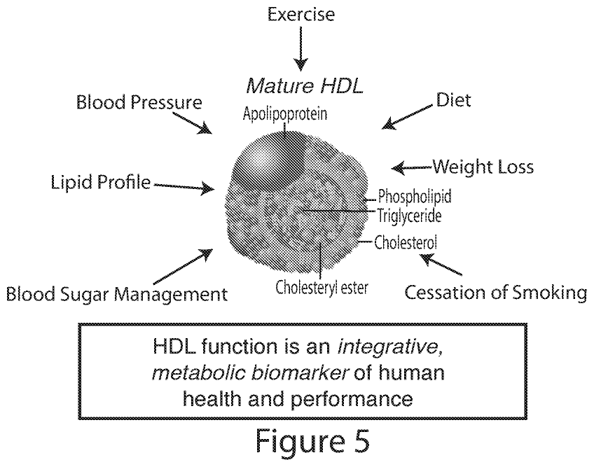

Measurements of serum cholesterol have been used to estimate cardiovascular disease (CVD) risk. More recently, the role of high-density lipoprotein (HDL) function has been investigated to improve upon CVD risk assessment. HDL function is an integrative, metabolic biomarker of human health and performance (see FIG. 5). The current test of HDL function is time-consuming, cost-prohibitive and not reproducible. A rapid and cost-effective assay is reported, measuring HDL function based upon nanoparticle sequestration of serum apolipoprotein A-I (apoAI) and cholesterol. The formed particles serve as a catalyst for cholesterol esterification by lecithin:cholesterol acyltransferase (LCAT). Ultimately, measuring cholesteryl ester is correlated to serum apoAI and HDL function.

### HDL-NP Support LCAT Activity

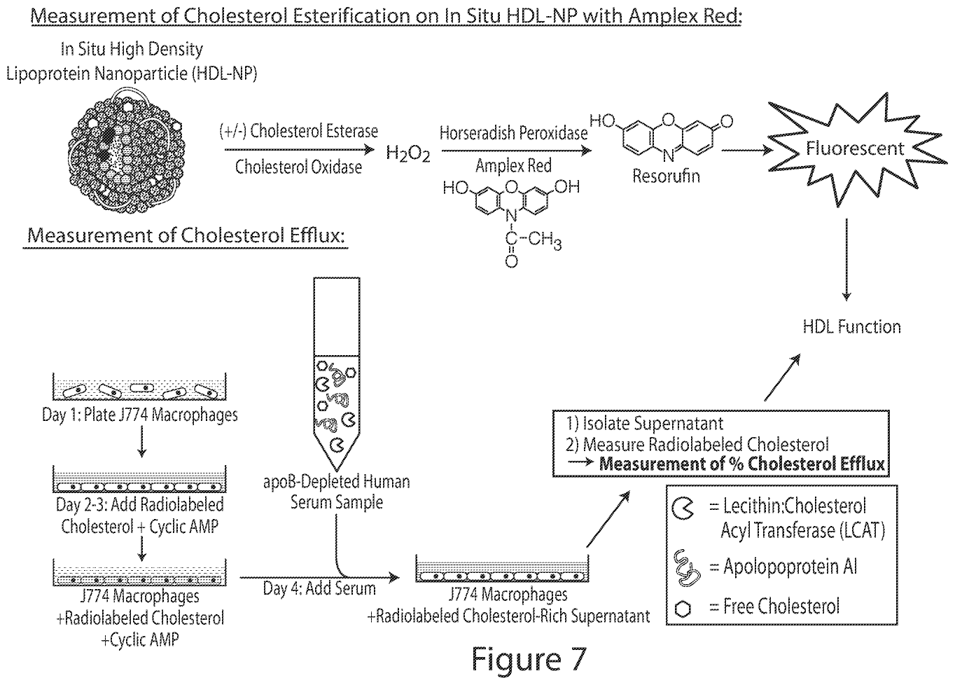

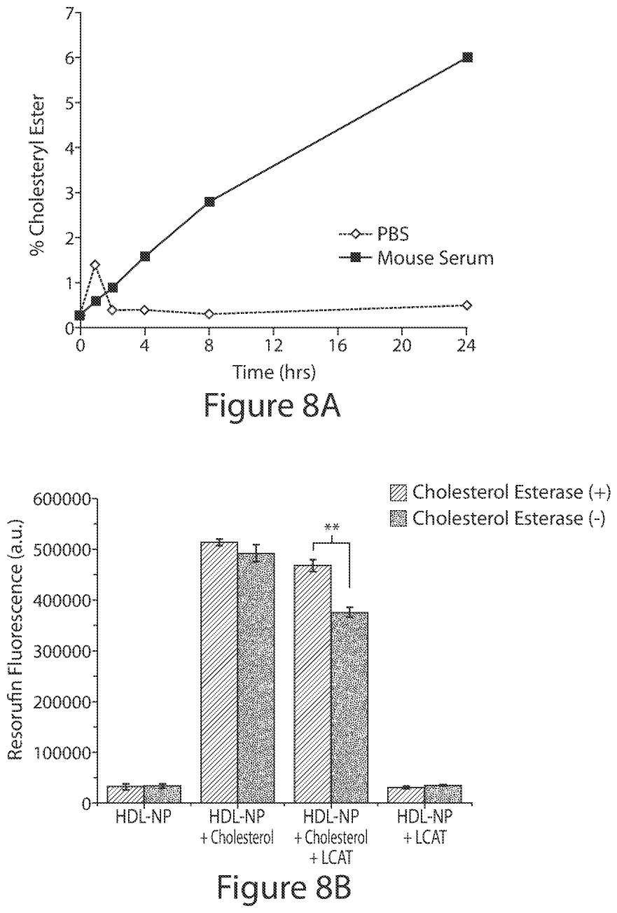

HDL-NP have been previously shown to support cholesterol loading and efflux from cells. The results in FIGS. 8A and 8B demonstrate their ability to support LCAT activity and cholesterol esterification. HDL-NP were incubated with cholesterol-laden macrophages to load cholesterol via cholesterol efflux, then purified and incubated with mouse serum as a source of LCAT. Incubation with mouse serum demonstrates a time-dependent increase in cholesteryl esters. (FIG. 8A) A similar experiment was conducted where HDL-NP were loaded with cholesterol and purified LCAT was added for 24 hours, after which cholesteryl esters were measured using an Amplex Red Assay kit. The measurement of cholesterol esterification on in situ HDL-NP with Amplex Red is shown in FIG. 7. This experiment demonstrates the specificity of LCAT activity on the HDL-NP.

ApoAI Sequestration with Bilayer NP

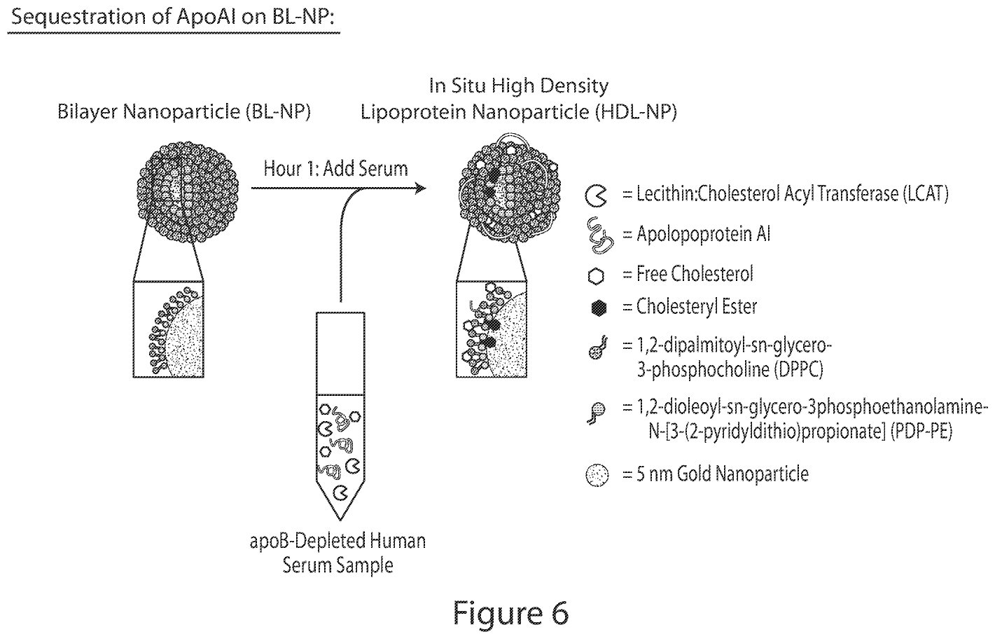

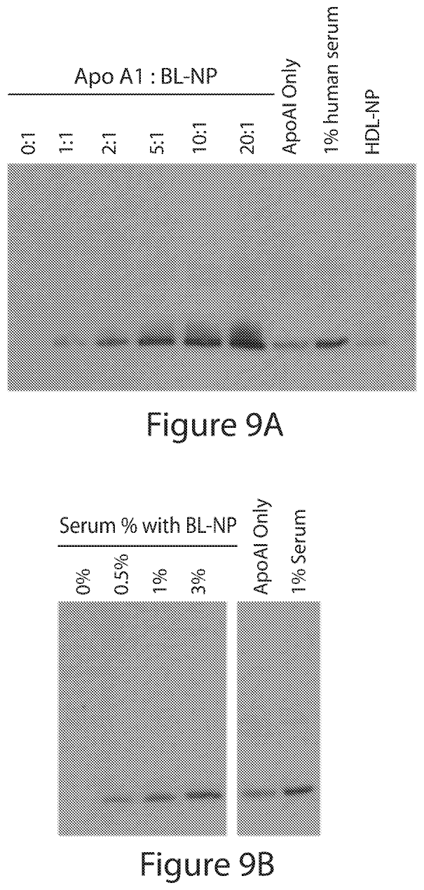

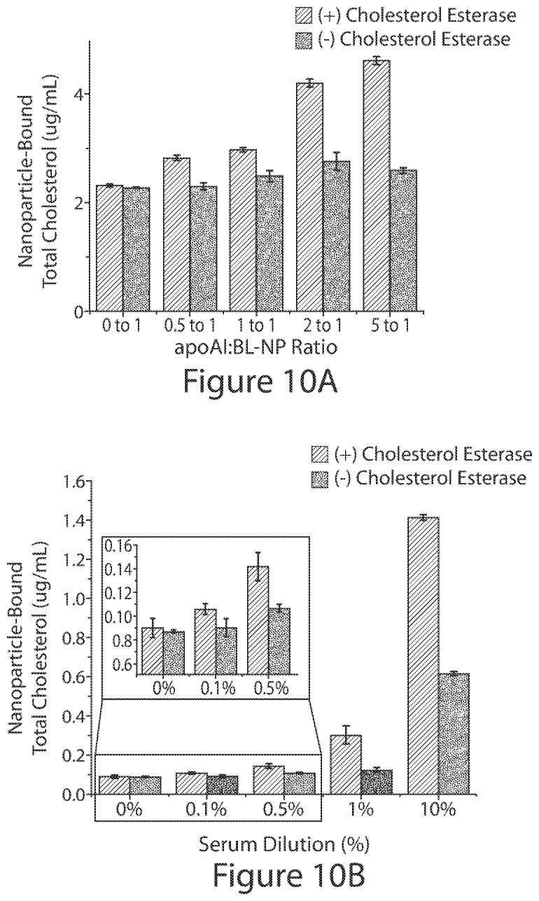

Bilayer NP were tested for their ability to sequester apoAI from solution (see FIG. 6). Following 1 hr of incubation of ApoAI with BL-NP, nanoparticles were purified and nanoparticle-associated ApoAI was assayed by Western Blot. A dose-dependent increase in nanoparticle-associated ApoAI was observed with increasing ApoAI:BL-NP ratio, demonstrating strong potential for apoAI sequestration by BL-NP (FIG. 9A). BL-NP were similarly incubated in serum to observe the amount of apoAI associated with the nanoparticles (FIG. 9B) and assayed for the formation of cholesteryl esters (FIGS. 10A and 10B). Incubation of BL-NP with either a mixture of apoAI/cholesterol/LCAT or serum once again demonstrates a dose-dependent apoAI adsorption and corresponding LCAT activity.

Correlation of BL-NP Assay with ApoAI Levels and HDL Function in Human Serum

Two cohorts of human serum samples were used to test BL-NP assay for relationship to serum apoAI levels and HDL cholesterol efflux (see FIG. 7).

Cohort A: Commercially available human serum, N=18
Cohort B: Chicago Healthy Aging Study serum samples, N=28

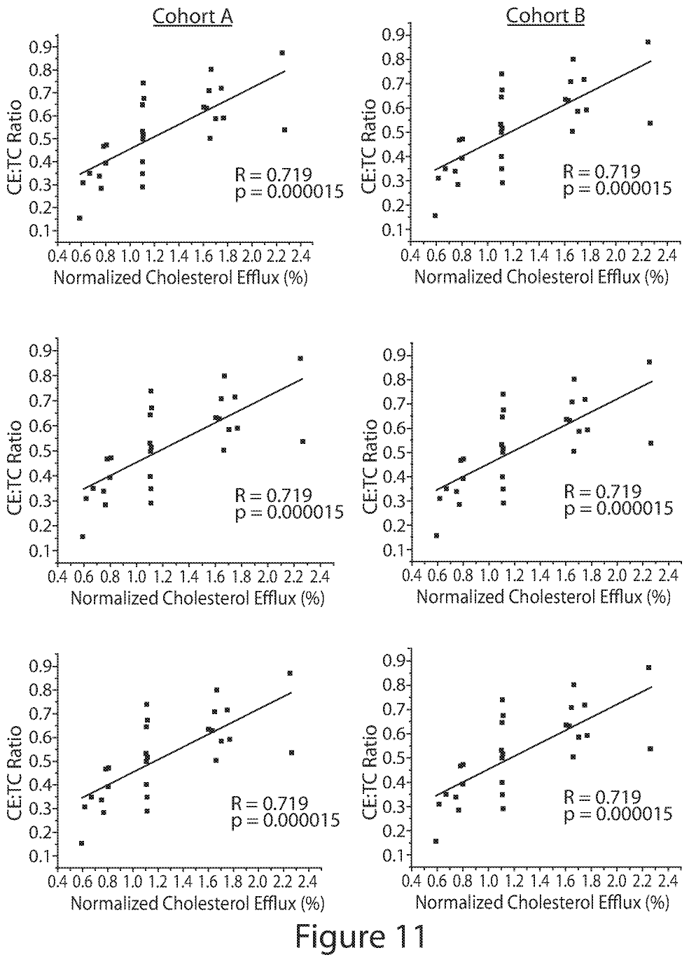

The output of BL-NP assay (Cholesterol Esters:Total Cholesterol ratio, CE:TC ratio) is positively and significantly correlated to HDL Cholesterol Efflux and serum apoAI (see FIG. 11), suggesting that the nanoparticle assay could be predictive of both metrics from one single measurement. Significant positive correlation between serum apoAI and HDL cholesterol efflux confirms the relationship between each metric.

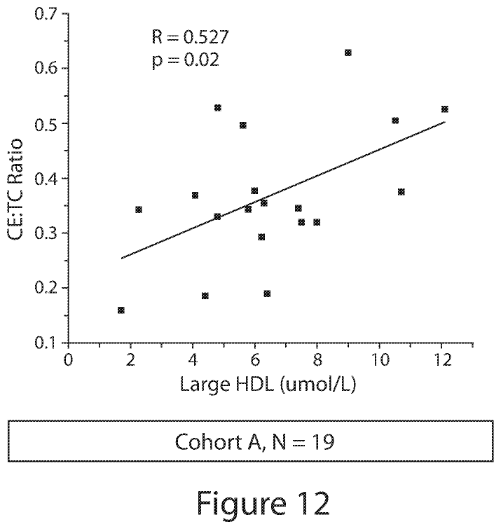

The amount of cholesterol esters on IS-HDL NP was examined. IT was found that the concentration of cholesterol esters correlates with the concentration of large HDL The data is shown in FIG. 12 (Cohort A, N=19).

### SUMMARY

BL-NP serve as promising candidates for development of biosensors for apoAI detection and assessment of HDL function. The use of these platforms allows for the potential development of rapid and simple systems for point-of-care measurement and evaluation of HDL function as it relates to cardiovascular disease risk.

### REFERENCES

(1) Thaxton, C. S.; Daniel, W. L.; Giljohann, D. A.; Thomas, A. D.; Mirkin, C. A. J. Am. Chem. Soc. 2009, 131 (4), 1384-1385.
(2) Lai, C.-T.; Sun, W.; Palekar, R. U.; Thaxton, C. S.; Schatz, G. C. ACS Appl Mater Interfaces 2017, 9 (2), 1247-1254.
(3) Luthi, A. J.; Zhang, H.; Kim, D.; Giljohann, D. A.; Mirkin, C. A.; Thaxton, C. S. ACS Nano 2012, 6 (1), 276-285.
(4) Frey, I.; Baumstark, M. W.; Berg, A.; Keul, J. European Journal of Applied Physiology and Occupational Physiology 1991, 62 (1), 31-35.
(5) Kraus, W. E.; Houmard, J. A.; Duscha, B. D.; Knetzger, K. J.; Wharton, M. B.; McCartney, J. S.; Bales, C. W.; Henes, S.; Samsa, G. P.; Otvos, J. D.; Kulkarni, K. R.; Slentz, C. A. N. Engl. J. Med. 2002, 347 (19), 1483-1492.
(6) Durstine, J. L.; Grandjean, P. W.; Davis, P. G.; Ferguson, M. A.; Alderson, N. L.; DuBose, K. D. Sports Medicine 2001, 31 (15), 1033-1062.
(7) Pronk, N. P. Sports Medicine 1993, 16 (6), 431-448.
(8) Jafari, M.; Leaf, D. A.; MacRae, H.; Kasem, J.; O'Conner, P.; Pullinger, C.; Malloy, M.; Kane, J. P. Metabolism 2003, 52 (4), 437-442.
(9) Sviridov, D.; Kingwell, B.; Hoang, A.; Dart, A.; Nestel, P. The Journal of Lipid Research 2003, 44 (3), 522-526.
(10) Olchawa, B.; Kingwell, B. A.; Hoang, A.; Schneider, L.; Miyazaki, O.; Nestel, P.; Sviridov, D. Arteriosclerosis, Thrombosis, and Vascular Biology 2004, 24 (6), 1087-1091
(11) McMahon, K. M.; Foit, L.; Angeloni, N. L.; Giles, F. J.; Gordon, L. I.; Thaxton, C. S. Cancer Treat. Res. 2015, 166, 129-150.
(12) Yang, S.; Damiano, M. G.; Zhang, H.; Tripathy, S.; Luthi, A. J.; Rink, J. S.; Ugolkov, A. V.; Singh, A. T. K.; Dave, S. S.; Gordon, L. I.; Thaxton, C. S. Proceedings of the National Academy of Sciences 2013, 110 (7), 2511-2516.
(13) Foit, L.; Thaxton, C. S. Biomaterials 2016, 100, 67-75.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention.

1. A biosensor for detecting and/or quantifying an exercise and disease-risk associated enzyme in a liquid sample, the biosensor comprising: (a) a solid support; and (b) a nanostructure comprising a metallic core of 1-10 nm in diameter and a phospholipid shell capable of binding to a lecithin:cholesterol acyltransferase (LCAT) activator (BL-NP), wherein the BL-NP is bound to the solid support.

2. The biosensor of claim 1, wherein the biosensor is a wearable device.

3. The biosensor of claim 1, wherein the biosensor is not a wearable device.

4. The biosensor of claim 1, wherein the solid support comprises plastic, glass, metal, metal oxide or crystal, and the solid support is in the form of a testing strip, a well in a microwell plate or a microcentrifuge tube; or (b) the solid support comprises or consists essentially of silica gel or other porous particle packing material for chromatography.

5. The biosensor of claim 1, wherein the phospholipid shell binds the LCAT activator.

6. The biosensor of claim 1, wherein the LCAT activator is an apolipoprotein.

7. The biosensor of claim 6, wherein the metallic core is a gold core.

8. The biosensor of claim 7, wherein the gold core has a diameter of 5 nm.

9. The biosensor of claim 7, wherein the gold core has a diameter of 4-6 nm.

10. The biosensor of claim 7, wherein the gold core has a diameter of 5-8 nm.

11. A method for rapid detection of an exercise and/or disease-risk associated enzyme comprising:
contacting a biological sample with a biosensor comprising: (a) a solid support; and (b) a nanostructure comprising a metallic core of 1-10 nm in diameter and a phospholipid shell capable of binding to a lecithin:cholesterol acyltransferase (LCAT) activator (BL-NP), wherein the BL-NP is bound to the solid support, incubating the BL-NP with the biological sample for at least 15 minutes so that the BL-NP can bind to the LCAT activator, measuring LCAT activation as an indicator of the presence of the exercise and/or disease associated enzyme in the biological sample.

12. The method of claim 11, wherein the biological sample is selected from the group consisting of blood, blood matrix, serum, plasma, sputum, cerebrospinal fluid, breath condensate, saliva, and tears.

13. The method of claim 11, wherein the LCAT activator is an apolipoprotein.

14. The method of claim 11, wherein the disease associated enzyme is indicative of cardiovascular disease.

15. The method of claim 11, wherein the biological sample is serum and 1-2 uL of serum is used.

16. The method of claim 11, wherein the quantity of cholesterol ester present in the biological sample is determined.

17. The method of claim 11, wherein the biological sample is isolated from a subject after exercise.

18. The method of claim 11, wherein the biological sample is isolated from a subject during exercise.

19. The method of claim 11, wherein the biological sample is isolated from a subject before exercise.

20. The method of claim 11, wherein the biological sample is isolated from a subject during a clinical trial.

21. The method of claim 11, wherein the biosensor is a lateral flow assay system.

22. The method of claim 11, wherein the biosensor is a paper assay system.

23. The method of claim 11, wherein the phospholipids of the biosensor are not fluorescently labeled.

24. The method of claim 11, wherein the biosensor provides an assessment of HDL function.
